(19) United States
(12) Patent Application Publication
(10) Pub. No.: US 2023/0310781 A1
(43) Pub. Date: Oct. 5, 2023

(54) SYSTEMS AND METHODS FOR DETERMINING A MASK RECOMMENDATION

(71) Applicant: ResMed Pty Ltd, Bella Vista, NSW (AU)

(72) Inventors: Jose Ricardo Dos Santos, San Diego, CA (US); Redmond Shouldice, Dublin (IE); Ian Andrew Law, Sydney (AU)

(21) Appl. No.: 18/023,091

(22) PCT Filed: Aug. 31, 2021
(86) PCT No.: PCT/US2021/048459

§ 371 Date: Feb. 24, 2023

Related U.S. Application Data
(60) Provisional application No. 63/072,467, filed on Aug. 31, 2020.

(51) Int. Cl. A61M 16/06; G16H 20/40

(52) U.S. Cl. CPC A61M 16/06; G16H 20/40; A61M 2016/0661; A61M 2016/003

(57) ABSTRACT

A method includes receiving data associated with a user during a sleep session. The received data is analyzed to determine, for a selected timeframe during the sleep session, whether the user is breathing through nostrils of the user. Based at least in part on a result of the analysis, a mask recommendation is communicated.

### CROSS-REFERENCE TO RELATED APPLICATIONS

This application claims the benefit of, and priority to, U.S. Provisional Patent Application No. 63/072,467 filed on Aug. 31, 2020, which is hereby incorporated by reference herein in its entirety.

### TECHNICAL FIELD

The present disclosure relates generally to systems and methods for determining an appropriate mask for a user, and more particularly, to systems and methods for determining a breathing habit of the user using a sleep session and recommending a mask based on the breathing habit.

### BACKGROUND

Many individuals suffer from sleep-related and/or respiratory disorders such as, for example, Periodic Limb Movement Disorder (PLMD), Restless Leg Syndrome (RLS), Sleep-Disordered Breathing (SDB), Obstructive Sleep Apnea (OSA), apneas, Cheyne-Stokes Respiration (CSR), respiratory insufficiency, Obesity Hyperventilation Syndrome (OHS), Chronic Obstructive Pulmonary Disease (COPD), Neuromuscular Disease (NMD), and chest wall disorders. These disorders are often treated using a respiratory therapy system. However, some users find such systems to be uncomfortable, difficult to use, expensive, aesthetically unappealing and/or fail to perceive the benefits associated with using the system. As a result, some users may elect not to use the respiratory therapy system diligently, absent a demonstration of the severity of their symptoms when respiratory therapy treatment is not used. Improving comfort can go a long way to increasing adherence to therapy. The present disclosure is directed to solving these and other problems.

### SUMMARY

According to some implementations of the present disclosure, a method includes receiving data associated with a user during a sleep session. The received data is analyzed to determine, for a selected timeframe during the sleep session, whether the user is breathing through nostrils of the user. Based at least in part on a result of the analysis, a mask recommendation is communicated.

According to some implementations of the present disclosure, a system for determining a breathing habit of a user includes a substrate coupled to nostrils of the user, a memory storing machine-readable instructions, and a control system including one or more processors configured to execute the machine-readable instructions to: receive, from the substrate, data associated with the user during a sleep session; analyze the received data to determine, for a selected timeframe, whether the user is breathing through the nostrils of the user; and based at least in part on a result of the analysis, causing a mask recommendation to be communicated to the user.

The above summary is not intended to represent each implementation or every aspect of the present disclosure. Additional features and benefits of the present disclosure are apparent from the detailed description and figures set forth below.

### BRIEF DESCRIPTION OF THE DRAWINGS

FIG. 1 is a functional block diagram of a system, according to some implementations of the present disclosure;

FIG. 2 is a perspective view of at least a portion of the system of FIG. 1, a user, and a bed partner, according to some implementations of the present disclosure;

FIG. 3 illustrates an exemplary timeline for a sleep session, according to some implementations of the present disclosure;

FIG. 4 is a process flow diagram for a method for providing a mask recommendation, according to some implementations of the present disclosure;

FIG. 5 illustrates an example of a substrate, according to some implementations of the present disclosure;

FIG. 6 illustrates an example of another substrate, according to some implementations of the present disclosure;

FIG. 7 illustrates an example of another substrate, according to some implementations of the present disclosure;

FIG. 8 illustrates an example of another substrate, according to some implementations of the present disclosure;

FIG. 9A illustrates a front view of an example substrate, according to some implementations of the present disclosure;

FIG. 9B illustrates a side view of the substrate of FIG. 9A;

FIG. 10A illustrates a side view of an example substrate, according to some implementations of the present disclosure;

FIG. 10B illustrates a front view of the substrate of FIG. 10A;

FIG. 11A illustrates a side view of an example substrate, according to some implementations of the present disclosure;

FIG. 11B illustrates a front view of the substrate of FIG. 11A;

FIG. 12A illustrates a side view of an example substrate, according to some implementations of the present disclosure; and

FIG. 12B illustrates a front view of the substrate of FIG. 12A.

While the present disclosure is susceptible to various modifications and alternative forms, specific implementations and embodiments thereof have been shown by way of example in the drawings and will herein be described in detail. It should be understood, however, that it is not intended to limit the present disclosure to the particular forms disclosed, but on the contrary, the present disclosure is to cover all modifications, equivalents, and alternatives falling within the spirit and scope of the present disclosure as defined by the appended claims.

### DETAILED DESCRIPTION

Many individuals suffer from sleep-related and/or respiratory disorders. Examples of sleep-related and/or respiratory disorders include Periodic Limb Movement Disorder (PLMD), Restless Leg Syndrome (RLS), Sleep-Disordered Breathing (SDB), Obstructive Sleep Apnea (OSA), apneas, Cheyne-Stokes Respiration (CSR), respiratory insufficiency, Obesity Hyperventilation Syndrome (OHS), Chronic Obstructive Pulmonary Disease (COPD), Neuromuscular Disease (NMD), and chest wall disorders.

Obstructive Sleep Apnea (OSA) is a form of Sleep Disordered Breathing (SDB), and is characterized by events including occlusion or obstruction of the upper air passage during sleep resulting from a combination of an abnormally small upper airway and the normal loss of muscle tone in the region of the tongue, soft palate and posterior oropharyngeal wall. More generally, an apnea generally refers to the cessation of breathing caused by blockage of the air (Obstructive Sleep Apnea) or the stopping of the breathing function (often referred to as central apnea). Typically, the individual will stop breathing for between about 15 seconds and about 30 seconds during an obstructive sleep apnea event.

Other types of apneas include hypopnea, hyperpnea, and hypercapnia. Hypopnea is generally characterized by slow or shallow breathing caused by a narrowed airway, as opposed to a blocked airway. Hyperpnea is generally characterized by an increase depth and/or rate of breathing. Hypercapnia is generally characterized by elevated or excessive carbon dioxide in the bloodstream, typically caused by inadequate respiration.

Cheyne-Stokes Respiration (CSR) is another form of sleep disordered breathing. CSR is a disorder of a patient's respiratory controller in which there are rhythmic alternating periods of waxing and waning ventilation known as CSR cycles. CSR is characterized by repetitive de-oxygenation and re-oxygenation of the arterial blood.

Obesity Hyperventilation Syndrome (OHS) is defined as the combination of severe obesity and awake chronic hypercapnia, in the absence of other known causes for hypoventilation. Symptoms include dyspnea, morning headache and excessive daytime sleepiness.

Chronic Obstructive Pulmonary Disease (COPD) encompasses any of a group of lower airway diseases that have certain characteristics in common, such as increased resistance to air movement, extended expiratory phase of respiration, and loss of the normal elasticity of the lung.

Neuromuscular Disease (NMD) encompasses many diseases and ailments that impair the functioning of the muscles either directly via intrinsic muscle pathology, or indirectly via nerve pathology. Chest wall disorders are a group of thoracic deformities that result in inefficient coupling between the respiratory muscles and the thoracic cage.

These and other disorders are characterized by particular events (e.g., snoring, an apnea, a hypopnea, a restless leg, a sleeping disorder, choking, an increased heart rate, labored breathing, an asthma attack, an epileptic episode, a seizure, or any combination thereof) that occur when the individual is sleeping.

The Apnea-Hypopnea Index (AHI) is an index used to indicate the severity of sleep apnea during a sleep session. The AHI is calculated by dividing the number of apnea and/or hypopnea events experienced by the user during the sleep session by the total number of hours of sleep in the sleep session. The event can be, for example, a pause in breathing that lasts for at least 10 seconds. An AHI that is less than 5 is considered normal. An AHI that is greater than or equal to 5, but less than 15 is considered indicative of mild sleep apnea. An AHI that is greater than or equal to 15, but less than 30 is considered indicative of moderate sleep apnea. An AHI that is greater than or equal to 30 is considered indicative of severe sleep apnea. In children, an AHI that is greater than 1 is considered abnormal. Sleep apnea can be considered “controlled” when the AHI is normal, or when the AHI is normal or mild. The AHI can also be used in combination with oxygen desaturation levels to indicate the severity of Obstructive Sleep Apnea.

When a user or a patient is prescribed to use a respiratory therapy system to alleviate symptoms related to any one of the respiratory conditions described above, the user may not know what type of user interface would be most appropriate for her. Some implementations of the present disclosure provide systems and methods for determining appropriate user interfaces using a breathing habit of the user. Outside of the environment of using a respiratory therapy system, nose-breathing is associated with many health benefits. For example, nostrils and sinuses filter and warm/cool air as the air enters the body. Sinuses produce nitric oxide, which when carried into the body through the breath, combats harmful bacterial and viruses, regulates blood pressure and boosts the immune system. Air breathed through the nose passes the nasal mucosa, which stimulates reflex nerves that control breathing, but this pathway is bypassed by mouth breathing which can lead to snoring, breath irregularities and sleep apnea. Nose breathing can force slowing down of the breath which can reduce hypertension and stress.

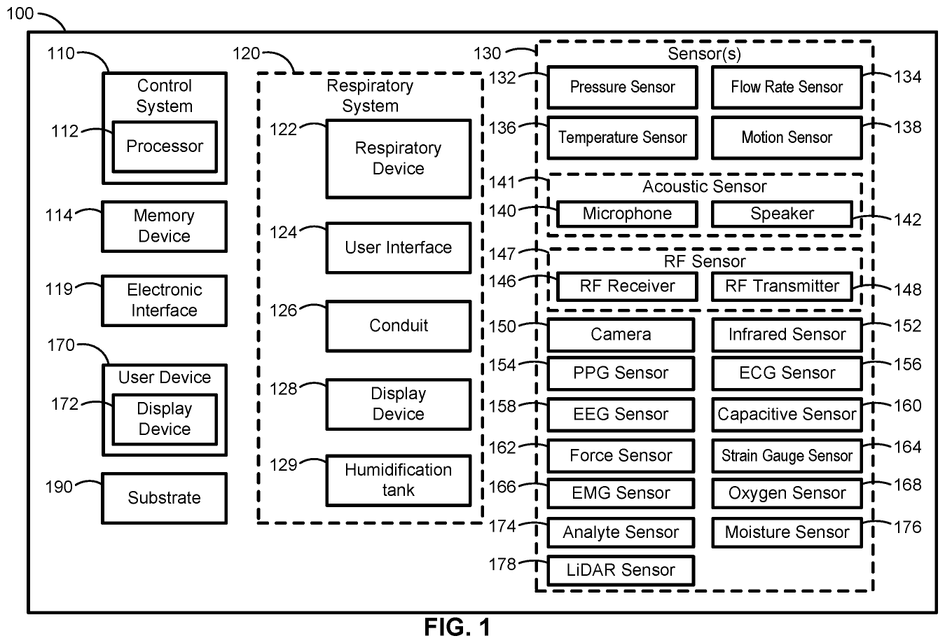

Referring to FIG. 1, a system 100, according to some implementations of the present disclosure, is illustrated. The system 100 includes a control system 110, a memory device 114, an electronic interface 119, one or more sensors 130, one or more user devices 170 and a substrate 190. In some implementations, the system 100 further optionally includes a respiratory therapy system 120.

The control system 110 includes one or more processors 112 (hereinafter, processor 112). The control system 110 is generally used to control (e.g., actuate) the various components of the system 100 and/or analyze data obtained and/or generated by the components of the system 100. The processor 112 can be a general or special purpose processor or microprocessor. While one processor 112 is shown in FIG. 1, the control system 110 can include any suitable number of processors (e.g., one processor, two processors, five processors, ten processors, etc.) that can be in a single housing, or located remotely from each other. The control system 110 can be coupled to and/or positioned within, for example, a housing of the user device 170, and/or within a housing of one or more of the sensors 130. The control system 110 can be centralized (within one such housing) or decentralized (within two or more of such housings, which are physically distinct). In such implementations including two or more housings containing the control system 110, such housings can be located proximately and/or remotely from each other.

The memory device 114 stores machine-readable instructions that are executable by the processor 112 of the control system 110. The memory device 114 can be any suitable computer readable storage device or media, such as, for example, a random or serial access memory device, a hard drive, a solid state drive, a flash memory device, etc. While one memory device 114 is shown in FIG. 1, the system 100 can include any suitable number of memory devices 114 (e.g., one memory device, two memory devices, five memory devices, ten memory devices, etc.). The memory device 114 can be coupled to and/or positioned within a housing of the respiratory therapy device 122, within a housing of the user device 170, within a housing of one or more of the sensors 130, or any combination thereof. Like the control system 110, the memory device 114 can be centralized (within one such housing) or decentralized (within two or more of such housings, which are physically distinct).

In some implementations, the memory device 114 (FIG. 1) stores a user profile associated with the user. The user profile can include, for example, demographic information associated with the user, biometric information associated with the user, medical information associated with the user, self-reported user feedback, sleep parameters associated with the user (e.g., sleep-related parameters recorded from one or more earlier sleep sessions), or any combination thereof. The demographic information can include, for example, information indicative of an age of the user, a gender of the user, a race of the user, a family history of insomnia, an employment status of the user, an educational status of the user, a socioeconomic status of the user, or any combination thereof. The medical information can include, for example, including indicative of one or more medical conditions associated with the user, medication usage by the user, or both. The medical information data can further include a multiple sleep latency test (MSLT) test result or score and/or a Pittsburgh Sleep Quality Index (PSQI) score or value. The self-reported user feedback can include information indicative of a self-reported subjective sleep score (e.g., poor, average, excellent), a self-reported subjective stress level of the user, a self-reported subjective fatigue level of the user, a self-reported subjective health status of the user, a recent life event experienced by the user, or any combination thereof.

The electronic interface 119 is configured to receive data (e.g., physiological data and/or audio data) from the one or more sensors 130 such that the data can be stored in the memory device 114 and/or analyzed by the processor 112 of the control system 110. The electronic interface 119 can communicate with the one or more sensors 130 using a wired connection or a wireless connection (e.g., using an RF communication protocol, a WiFi communication protocol, a Bluetooth communication protocol, over a cellular network, etc.). The electronic interface 119 can include an antenna, a receiver (e.g., an RF receiver), a transmitter (e.g., an RF transmitter), a transceiver, or any combination thereof. The electronic interface 119 can also include one more processors and/or one more memory devices that are the same as, or similar to, the processor 112 and the memory device 114 described herein. In some implementations, the electronic interface 119 is coupled to or integrated in the user device 170 and/or the substrate 190. In other implementations, the electronic interface 119 is coupled to or integrated (e.g., in a housing) with the control system 110 and/or the memory device 114.

The substrate 190 is an electronic device that can be coupled to a nose of the user. The substrate 190 can be affixed to the nose of the user using an adhesive. The substrate 190 can be pinched to the septum of the nose. The substrate 190 can be pinched to the bridge of the nose. The substrate 190 can be connected to straps and tied behind the head of the user. The substrate 190 can include at least one of the one or more sensors 130. The substrate 190 can include a small cell battery and can monitor breathing of the user to determine whether the user is breathing through the nose. In some implementations, the substrate 190 can be used to determine whether the user is breathing through the mouth. The substrate 190 can communicate with the user device 170.

As noted above, in some implementations, the system 100 optionally includes a respiratory therapy system 120. The respiratory therapy system 120 can include a respiratory pressure therapy device 122 (referred to herein as respiratory therapy device 122), a user interface 124, a conduit 126 (also referred to as a tube or an air circuit), a display device 128, a humidification tank 129, or any combination thereof. In some implementations, the control system 110, the memory device 114, the display device 128, one or more of the sensors 130, and the humidification tank 129 are part of the respiratory therapy device 122. Respiratory pressure therapy refers to the application of a supply of air to an entrance to a user's airways at a controlled target pressure that is nominally positive with respect to atmosphere throughout the user's breathing cycle (e.g., in contrast to negative pressure therapies such as the tank ventilator or cuirass). The respiratory therapy system 120 is generally used to treat individuals suffering from one or more sleep-related respiratory disorders (e.g., obstructive sleep apnea, central sleep apnea, or mixed sleep apnea).

The respiratory therapy device 122 is generally used to generate pressurized air that is delivered to a user (e.g., using one or more motors that drive one or more compressors). In some implementations, the respiratory therapy device 122 generates continuous constant air pressure that is delivered to the user. In other implementations, the respiratory therapy device 122 generates two or more predetermined pressures (e.g., a first predetermined air pressure and a second predetermined air pressure). In still other implementations, the respiratory therapy device 122 is configured to generate a variety of different air pressures within a predetermined range. For example, the respiratory therapy device 122 can deliver at least about 6 cm H₂O, at least about 10 cm H₂O, at least about 20 cm H₂O, between about 6 cm H₂O and about 10 cm H₂O, between about 7 cm H₂O and about 12 cm H₂O, etc. The respiratory therapy device 122 can also deliver pressurized air at a predetermined flow rate between, for example, about −20 L/min and about 150 L/min, while maintaining a positive pressure (relative to the ambient pressure).

The user interface 124 engages a portion of the user's face and delivers pressurized air from the respiratory therapy device 122 to the user's airway to aid in preventing the airway from narrowing and/or collapsing during sleep. This may also increase the user's oxygen intake during sleep. Depending upon the therapy to be applied, the user interface 124 may form a seal, for example, with a region or portion of the user's face, to facilitate the delivery of gas at a pressure at sufficient variance with ambient pressure to effect therapy, for example, at a positive pressure of about 10 cm H₂O relative to ambient pressure. For other forms of therapy, such as the delivery of oxygen, the user interface may not include a seal sufficient to facilitate delivery to the airways of a supply of gas at a positive pressure of about 10 cm H₂O.

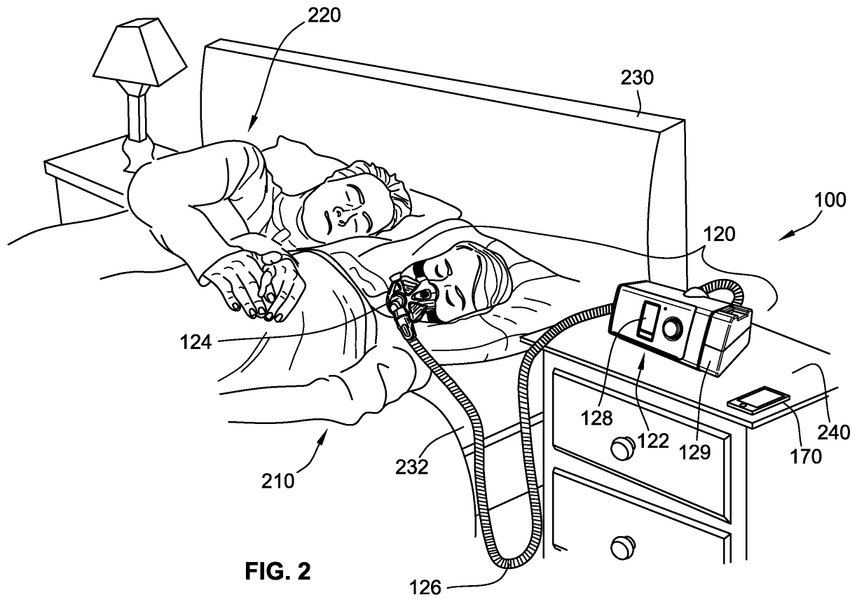

As shown in FIG. 2, in some implementations, the user interface 124 is a facial mask that covers the nose and mouth of the user. Alternatively, the user interface 124 can be a nasal mask that provides air to the nose of the user or a nasal pillow mask that delivers air directly to the nostrils of the user. The user interface 124 can include a plurality of straps (e.g., including hook and loop fasteners) for positioning and/or stabilizing the interface on a portion of the user (e.g., the face) and a conformal cushion (e.g., silicone, plastic, foam, etc.) that aids in providing an air-tight seal between the user interface 124 and the user. The user interface 124 can also include one or more vents for permitting the escape of carbon dioxide and other gases exhaled by the user 210. In other implementations, the user interface 124 is a mouthpiece (e.g., a night guard mouthpiece molded to conform to the user's teeth, a mandibular repositioning device, etc.) for directing pressurized air into the mouth of the user. The type of user interface 124 used can be more effective for certain users. For example, a user that breaths mostly through her mouth will not benefit much from a user interface that covers only the nose. As such, a facial mask covering the nose and mouth would be more beneficial to the user.

The conduit 126 (also referred to as an air circuit or tube) allows the flow of air between two components of a respiratory therapy system 120, such as the respiratory therapy device 122 and the user interface 124. In some implementations, there can be separate limbs of the conduit for inhalation and exhalation. In other implementations, a single limb conduit is used for both inhalation and exhalation.

One or more of the substrate 190, the respiratory therapy device 122, the user interface 124, the conduit 126, the display device 128, and the humidification tank 129 can contain one or more sensors (e.g., a pressure sensor, a flow rate sensor, or more generally any of the other sensors 130 described herein). These one or more sensors can be use, for example, to measure the air pressure and/or flow rate of pressurized air supplied by the respiratory therapy device 122.

The display device 128 is generally used to display image(s) including still images, video images, or both and/or information regarding the respiratory therapy device 122. For example, the display device 128 can provide information regarding the status of the respiratory therapy device 122 (e.g., whether the respiratory therapy device 122 is on/off, the pressure of the air being delivered by the respiratory therapy device 122, the temperature of the air being delivered by the respiratory therapy device 122, etc.) and/or other information (e.g., a sleep score, the current date/time, personal information for the user 210, etc.). In some implementations, the display device 128 acts as a human-machine interface (HMI) that includes a graphic user interface (GUI) configured to display the image(s) as an input interface. The display device 128 can be an LED display, an OLED display, an LCD display, or the like. The input interface can be, for example, a touchscreen or touch-sensitive substrate, a mouse, a keyboard, or any sensor system configured to sense inputs made by a human user interacting with the respiratory therapy device 122.

The humidification tank 129 is coupled to or integrated in the respiratory therapy device 122 and includes a reservoir of water that can be used to humidify the pressurized air delivered from the respiratory therapy device 122. The respiratory therapy device 122 can include a heater to heat the water in the humidification tank 129 in order to humidify the pressurized air provided to the user. Additionally, in some implementations, the conduit 126 can also include a heating element (e.g., coupled to and/or imbedded in the conduit 126) that heats the pressurized air delivered to the user.

The respiratory therapy system 120 can be used, for example, as a positive airway pressure (PAP) system, a continuous positive airway pressure (CPAP) system, an automatic positive airway pressure system (APAP), a bi-level or variable positive airway pressure system (BPAP or VPAP), a ventilator, or any combination thereof. The CPAP system delivers a predetermined air pressure (e.g., determined by a sleep physician) to the user. The APAP system automatically varies the air pressure delivered to the user based on, for example, respiration data associated with the user. The BPAP or VPAP system is configured to deliver a first predetermined pressure (e.g., an inspiratory positive airway pressure or IPAP) and a second predetermined pressure (e.g., an expiratory positive airway pressure or EPAP) that is lower than the first predetermined pressure.

Referring to FIG. 2, a portion of the system 100 (FIG. 1), according to some implementations, is illustrated. A user 210 of the respiratory therapy system 120 and a bed partner 220 are located in a bed 230 and are laying on a mattress 232. The user interface 124 (e.g., a full facial mask) can be worn by the user 210 during a sleep session. The user interface 124 is fluidly coupled and/or connected to the respiratory therapy device 122 via the conduit 126. In turn, the respiratory therapy device 122 delivers pressurized air to the user 210 via the conduit 126 and the user interface 124 to increase the air pressure in the throat of the user 210 to aid in preventing the airway from closing and/or narrowing during sleep. The respiratory therapy device 122 can be positioned on a nightstand 240 that is directly adjacent to the bed 230 as shown in FIG. 2, or more generally, on any surface or structure that is generally adjacent to the bed 230 and/or the user 210.

Referring to back to FIG. 1, the one or more sensors 130 of the system 100 include a pressure sensor 132, a flow rate sensor 134, temperature sensor 136, a motion sensor 138, a microphone 140, a speaker 142, a radio-frequency (RF) receiver 146, a RF transmitter 148, a camera 150, an infrared sensor 152, a photoplethysmogram (PPG) sensor 154, an electrocardiogram (ECG) sensor 156, an electroencephalography (EEG) sensor 158, a capacitive sensor 160, a force sensor 162, a strain gauge sensor 164, an electromyography (EMG) sensor 166, an oxygen sensor 168, an analyte sensor 174, a moisture sensor 176, a LiDAR sensor 178, or any combination thereof. Generally, each of the one or sensors 130 are configured to output sensor data that is received and stored in the memory device 114 or one or more other memory devices.

While the one or more sensors 130 are shown and described as including each of the pressure sensor 132, the flow rate sensor 134, the temperature sensor 136, the motion sensor 138, the microphone 140, the speaker 142, the RF receiver 146, the RF transmitter 148, the camera 150, the infrared sensor 152, the photoplethysmogram (PPG) sensor 154, the electrocardiogram (ECG) sensor 156, the electroencephalography (EEG) sensor 158, the capacitive sensor 160, the force sensor 162, the strain gauge sensor 164, the electromyography (EMG) sensor 166, the oxygen sensor 168, the analyte sensor 174, the moisture sensor 176, and the LiDAR sensor 178, more generally, the one or more sensors 130 can include any combination and any number of each of the sensors described and/or shown herein.

The one or more sensors 130 can be used to generate, for example, physiological data, audio data, or both. Physiological data generated by one or more of the sensors 130 can be used by the control system 110 to determine a sleep-wake signal associated with a user during a sleep session and one or more sleep-related parameters. The sleep-wake signal can be indicative of one or more sleep states, including wakefulness, relaxed wakefulness, micro-awakenings, a rapid eye movement (REM) stage, a first non-REM stage (often referred to as “N1”), a second non-REM stage (often referred to as “N2”), a third non-REM stage (often referred to as “N3”), or any combination thereof. The sleep-wake signal can also be timestamped to indicate a time that the user enters the bed, a time that the user exits the bed, a time that the user attempts to fall asleep, etc. The sleep-wake signal can be measured by the sensor(s) 130 during the sleep session at a predetermined sampling rate, such as, for example, one sample per second, one sample per 30 seconds, one sample per minute, etc. Examples of the one or more sleep-related parameters that can be determined for the user during the sleep session based on the sleep-wake signal include a total time in bed, a total sleep time, a sleep onset latency, a wake-after-sleep-onset parameter, a sleep efficiency, a fragmentation index, or any combination thereof.

Physiological data and/or audio data generated by the one or more sensors 130 can also be used to determine a respiration signal associated with a user during a sleep session. The respiration signal is generally indicative of respiration or breathing of the user during the sleep session. The respiration signal can be indicative of, for example, a respiration rate, a respiration rate variability, an inspiration amplitude, an expiration amplitude, an inspiration-expiration ratio, a number of events per hour, a pattern of events, pressure settings of the respiratory therapy device 122, or any combination thereof. The event(s) can include snoring, apneas, central apneas, obstructive apneas, mixed apneas, hypopneas, a mask leak (e.g., from the user interface 124), a restless leg, a sleeping disorder, choking, an increased heart rate, labored breathing, an asthma attack, an epileptic episode, a seizure, or any combination thereof.

The pressure sensor 132 outputs pressure data that can be stored in the memory device 114 and/or analyzed by the processor 112 of the control system 110. In some implementations, the pressure sensor 132 is an air pressure sensor (e.g., barometric pressure sensor) that generates sensor data indicative of the respiration (e.g., inhaling and/or exhaling) of the user of the respiratory therapy system 120 and/or ambient pressure. In such implementations, the pressure sensor 132 can be coupled to or integrated in the respiratory therapy device 122. The pressure sensor 132 can be, for example, a capacitive sensor, an electromagnetic sensor, a piezoelectric sensor, a strain-gauge sensor, an optical sensor, a potentiometric sensor, or any combination thereof.

The flow rate sensor 134 outputs flow rate data that can be stored in the memory device 114 and/or analyzed by the processor 112 of the control system 110. In some implementations, the flow rate sensor 134 is used to determine an air flow rate from the respiratory therapy device 122, an air flow rate through the conduit 126, an air flow rate through the user interface 124, or any combination thereof. In such implementations, the flow rate sensor 134 can be coupled to or integrated in the respiratory therapy device 122, the user interface 124, or the conduit 126. The flow rate sensor 134 can be a mass flow rate sensor such as, for example, a rotary flow meter (e.g., Hall effect flow meters), a turbine flow meter, an orifice flow meter, an ultrasonic flow meter, a hot wire sensor, a vortex sensor, a membrane sensor, or any combination thereof.

The temperature sensor 136 outputs temperature data that can be stored in the memory device 114 and/or analyzed by the processor 112 of the control system 110. In some implementations, the temperature sensor 136 generates temperatures data indicative of a core body temperature of the user 210 (FIG. 2), a skin temperature of the user 210, a temperature of the air flowing from the respiratory therapy device 122 and/or through the conduit 126, a temperature in the user interface 124, an ambient temperature, or any combination thereof. The temperature sensor 136 can be, for example, a thermocouple sensor, a thermistor sensor, a silicon band gap temperature sensor or semiconductor-based sensor, a resistance temperature detector, or any combination thereof.

The microphone 140 outputs audio data that can be stored in the memory device 114 and/or analyzed by the processor 112 of the control system 110. The audio data generated by the microphone 140 is reproducible as one or more sound(s) during a sleep session (e.g., sounds from the user 210). The audio data form the microphone 140 can also be used to identify (e.g., using the control system 110) an event experienced by the user during the sleep session, as described in further detail herein. The microphone 140 can be coupled to or integrated in the respiratory therapy device 122, the user interface 124, the conduit 126, or the user device 170.

The speaker 142 outputs sound waves that are audible to a user of the system 100 (e.g., the user 210 of FIG. 2). The speaker 142 can be used, for example, as an alarm clock or to play an alert or message to the user 210 (e.g., in response to an event). In some implementations, the speaker 142 can be used to communicate the audio data generated by the microphone 140 to the user. The speaker 142 can be coupled to or integrated in the respiratory therapy device 122, the user interface 124, the conduit 126, or the user device 170.

The microphone 140 and the speaker 142 can be used as separate devices. In some implementations, the microphone 140 and the speaker 142 can be combined into an acoustic sensor 141, as described in, for example, WO 2018/050913, which is hereby incorporated by reference herein in its entirety. In such implementations, the speaker 142 generates or emits sound waves at a predetermined interval and the microphone 140 detects the reflections of the emitted sound waves from the speaker 142. The sound waves generated or emitted by the speaker 142 have a frequency that is not audible to the human ear (e.g., below 20 Hz or above around 18 kHz) so as not to disturb the sleep of the user 210 or the bed partner 220 (FIG. 2). Based at least in part on the data from the microphone 140 and/or the speaker 142, the control system 110 can determine a location of the user 210 (FIG. 2) and/or one or more of the sleep-related parameters described in herein.

In some implementations, the sensors 130 include (i) a first microphone that is the same as, or similar to, the microphone 140, and is integrated in the acoustic sensor 141 and (ii) a second microphone that is the same as, or similar to, the microphone 140, but is separate and distinct from the first microphone that is integrated in the acoustic sensor 141.

The RF transmitter 148 generates and/or emits radio waves having a predetermined frequency and/or a predetermined amplitude (e.g., within a high frequency band, within a low frequency band, long wave signals, short wave signals, etc.). The RF receiver 146 detects the reflections of the radio waves emitted from the RF transmitter 148, and this data can be analyzed by the control system 110 to determine a location of the user 210 (FIG. 2) and/or one or more of the sleep-related parameters described herein. An RF receiver (either the RF receiver 146 and the RF transmitter 148 or another RF pair) can also be used for wireless communication between the control system 110, the respiratory therapy device 122, the one or more sensors 130, the user device 170, or any combination thereof. While the RF receiver 146 and RF transmitter 148 are shown as being separate and distinct elements in FIG. 1, in some implementations, the RF receiver 146 and RF transmitter 148 are combined as a part of an RF sensor 147. In some such implementations, the RF sensor 147 includes a control circuit. The specific format of the RF communication can be WiFi, Bluetooth, or the like.

In some implementations, the RF sensor 147 is a part of a mesh system. One example of a mesh system is a WiFi mesh system, which can include mesh nodes, mesh router(s), and mesh gateway(s), each of which can be mobile/movable or fixed. In such implementations, the WiFi mesh system includes a WiFi router and/or a WiFi controller and one or more satellites (e.g., access points), each of which include an RF sensor that the is the same as, or similar to, the RF sensor 147. The WiFi router and satellites continuously communicate with one another using WiFi signals. The WiFi mesh system can be used to generate motion data based on changes in the WiFi signals (e.g., differences in received signal strength) between the router and the satellite(s) due to an object or person moving partially obstructing the signals. The motion data can be indicative of motion, breathing, heart rate, gait, falls, behavior, etc., or any combination thereof.

The camera 150 outputs image data reproducible as one or more images (e.g., still images, video images, thermal images, or a combination thereof) that can be stored in the memory device 114. The image data from the camera 150 can be used by the control system 110 to determine one or more of the sleep-related parameters described herein. For example, the image data from the camera 150 can be used to identify a location of the user, to determine a time when the user 210 enters the bed 230 (FIG. 2), and to determine a time when the user 210 exits the bed 230.

The infrared (IR) sensor 152 outputs infrared image data reproducible as one or more infrared images (e.g., still images, video images, or both) that can be stored in the memory device 114. The infrared data from the IR sensor 152 can be used to determine one or more sleep-related parameters during a sleep session, including a temperature of the user 210 and/or movement of the user 210. The IR sensor 152 can also be used in conjunction with the camera 150 when measuring the presence, location, and/or movement of the user 210. The IR sensor 152 can detect infrared light having a wavelength between about 700 nm and about 1 mm, for example, while the camera 150 can detect visible light having a wavelength between about 380 nm and about 740 nm.

The PPG sensor 154 outputs physiological data associated with the user 210 (FIG. 2) that can be used to determine one or more sleep-related parameters, such as, for example, a heart rate, a heart rate variability, a cardiac cycle, respiration rate, an inspiration amplitude, an expiration amplitude, an inspiration-expiration ratio, estimated blood pressure parameter(s), or any combination thereof. The PPG sensor 154 can be worn by the user 210, embedded in clothing and/or fabric that is worn by the user 210, embedded in and/or coupled to the user interface 124 and/or its associated headgear (e.g., straps, etc.), etc.

The ECG sensor 156 outputs physiological data associated with electrical activity of the heart of the user 210. In some implementations, the ECG sensor 156 includes one or more electrodes that are positioned on or around a portion of the user 210 during the sleep session. The physiological data from the ECG sensor 156 can be used, for example, to determine one or more of the sleep-related parameters described herein.

The EEG sensor 158 outputs physiological data associated with electrical activity of the brain of the user 210. In some implementations, the EEG sensor 158 includes one or more electrodes that are positioned on or around the scalp of the user 210 during the sleep session. The physiological data from the EEG sensor 158 can be used, for example, to determine a sleep state of the user 210 at any given time during the sleep session. In some implementations, the EEG sensor 158 can be integrated in the user interface 124 and/or the associated headgear (e.g., straps, etc.).

The capacitive sensor 160, the force sensor 162, and the strain gauge sensor 164 output data that can be stored in the memory device 114 and used by the control system 110 to determine one or more of the sleep-related parameters described herein. The EMG sensor 166 outputs physiological data associated with electrical activity produced by one or more muscles. The oxygen sensor 168 outputs oxygen data indicative of an oxygen concentration of gas (e.g., in the conduit 126 or at the user interface 124). The oxygen sensor 168 can be, for example, an ultrasonic oxygen sensor, an electrical oxygen sensor, a chemical oxygen sensor, an optical oxygen sensor, or any combination thereof. In some implementations, the one or more sensors 130 also include a galvanic skin response (GSR) sensor, a blood flow sensor, a respiration sensor, a pulse sensor, a sphygmomanometer sensor, an oximetry sensor, or any combination thereof.

The analyte sensor 174 can be used to detect the presence of an analyte in the exhaled breath of the user 210. The data output by the analyte sensor 174 can be stored in the memory device 114 and used by the control system 110 to determine the identity and concentration of any analytes in the breath of the user 210. In some implementations, the analyte sensor 174 is positioned near a mouth of the user 210 to detect analytes in breath exhaled from the user 210's mouth. For example, when the user interface 124 is a facial mask that covers the nose and mouth of the user 210, the analyte sensor 174 can be positioned within the facial mask to monitor the user 210's mouth breathing. In other implementations, such as when the user interface 124 is a nasal mask or a nasal pillow mask, the analyte sensor 174 can be positioned near the nose of the user 210 to detect analytes in breath exhaled through the user's nose. In still other implementations, the analyte sensor 174 can be positioned near the user 210's mouth when the user interface 124 is a nasal mask or a nasal pillow mask. In this implementation, the analyte sensor 174 can be used to detect whether any air is inadvertently leaking from the user 210's mouth. In some implementations, the analyte sensor 174 is a volatile organic compound (VOC) sensor that can be used to detect carbon-based chemicals or compounds. In some implementations, the analyte sensor 174 can also be used to detect whether the user 210 is breathing through their nose or mouth. For example, if the data output by an analyte sensor 174 positioned near the mouth of the user 210 or within the facial mask (in implementations where the user interface 124 is a facial mask) detects the presence of an analyte, the control system 110 can use this data as an indication that the user 210 is breathing through their mouth.

The moisture sensor 176 outputs data that can be stored in the memory device 114 and used by the control system 110. The moisture sensor 176 can be used to detect moisture in various areas surrounding the user (e.g., inside the conduit 126 or the user interface 124, near the user 210's face, near the connection between the conduit 126 and the user interface 124, near the connection between the conduit 126 and the respiratory therapy device 122, etc.). Thus, in some implementations, the moisture sensor 176 can be coupled to or integrated in the user interface 124 or in the conduit 126 to monitor the humidity of the pressurized air from the respiratory therapy device 122. In other implementations, the moisture sensor 176 is placed near any area where moisture levels need to be monitored. The moisture sensor 176 can also be used to monitor the humidity of the ambient environment surrounding the user 210, for example, the air inside the bedroom.

The Light Detection and Ranging (LiDAR) sensor 178 can be used for depth sensing. This type of optical sensor (e.g., laser sensor) can be used to detect objects and build three dimensional (3D) maps of the surroundings, such as of a living space. LiDAR can generally utilize a pulsed laser to make time of flight measurements. LiDAR is also referred to as 3D laser scanning. In an example of use of such a sensor, a fixed or mobile device (such as a smartphone) having a LiDAR sensor 166 can measure and map an area extending 5 meters or more away from the sensor. The LiDAR data can be fused with point cloud data estimated by an electromagnetic RADAR sensor, for example. The LiDAR sensor 178 can also use artificial intelligence (AI) to automatically geofence RADAR systems by detecting and classifying features in a space that might cause issues for RADAR systems, such as glass windows (which can be highly reflective to RADAR). LiDAR can also be used to provide an estimate of the height of a person, as well as changes in height when the person sits down, or falls down, for example. LiDAR may be used to form a 3D mesh representation of an environment. In a further use, for solid surfaces through which radio waves pass (e.g., radio-translucent materials), the LiDAR may reflect off such surfaces, thus allowing a classification of different type of obstacles.

While shown separately in FIG. 1, any combination of the one or more sensors 130 can be integrated in and/or coupled to any one or more of the components of the system 100, including the respiratory therapy device 122, the user interface 124, the conduit 126, the humidification tank 129, the control system 110, the user device 170, the substrate 190, or any combination thereof. For example, the microphone 140 and speaker 142 is integrated in and/or coupled to the user device 170 and the pressure sensor 130, flow rate sensor 132, and/or the temperature sensor 136 are integrated in and/or coupled to the substrate 190. In some implementations, at least one of the one or more sensors 130 is not coupled to the respiratory therapy device 122, the control system 110, or the user device 170, and is positioned generally adjacent to the user 210 during the sleep session (e.g., positioned on or in contact with a portion of the user 210, worn by the user 210, coupled to or positioned on the nightstand, coupled to the mattress, coupled to the ceiling, etc.).

The user device 170 (FIG. 1) includes a display device 172. The user device 170 can be, for example, a mobile device such as a smart phone, a tablet, a laptop, or the like. Alternatively, the user device 170 can be an external sensing system, a television (e.g., a smart television) or another smart home device (e.g., a smart speaker(s) such as Google Home, Amazon Echo, Alexa etc.). In some implementations, the user device is a wearable device (e.g., a smart watch). The display device 172 is generally used to display image(s) including still images, video images, or both. In some implementations, the display device 172 acts as a human-machine interface (HMI) that includes a graphic user interface (GUI) configured to display the image(s) and an input interface. The display device 172 can be an LED display, an OLED display, an LCD display, or the like. The input interface can be, for example, a touchscreen or touch-sensitive substrate, a mouse, a keyboard, or any sensor system configured to sense inputs made by a human user interacting with the user device 170. In some implementations, one or more user devices can be used by and/or included in the system 100.

While the control system 110 and the memory device 114 are described and shown in FIG. 1 as being a separate and distinct component of the system 100, in some implementations, the control system 110 and/or the memory device 114 are integrated in the user device 170, the substrate 190, and/or the respiratory therapy device 122. Alternatively, in some implementations, the control system 110 or a portion thereof (e.g., the processor 112) can be located in a cloud (e.g., integrated in a server, integrated in an Internet of Things (IoT) device, connected to the cloud, be subject to edge cloud processing, etc.), located in one or more servers (e.g., remote servers, local servers, etc., or any combination thereof.

While system 100 is shown as including all of the components described above, more or fewer components can be included in a system for generating physiological data and determining a recommended notification or action for the user according to implementations of the present disclosure. For example, a first alternative system includes the control system 110, the memory device 114, and at least one of the one or more sensors 130. As another example, a second alternative system includes the control system 110, the memory device 114, at least one of the one or more sensors 130, and the user device 170. As yet another example, a third alternative system includes the control system 110, the memory device 114, at least one of the one or more sensors 130, and the substrate 190. Thus, various systems can be formed using any portion or portions of the components shown and described herein and/or in combination with one or more other components.

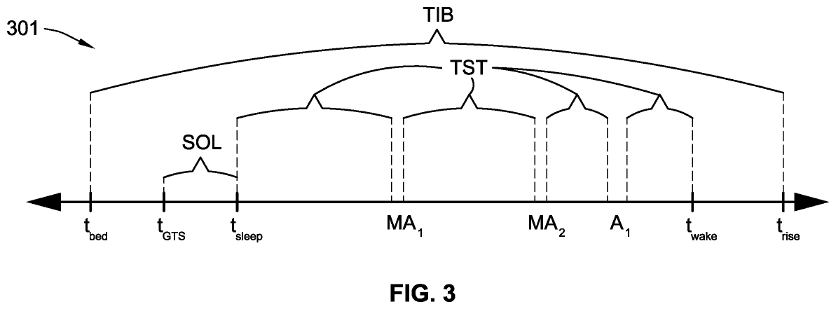

As used herein, a sleep session can be defined in a number of ways based on, for example, an initial start time and an end time. Referring to FIG. 3, an exemplary timeline 300 for a sleep session is illustrated. The timeline 300 includes an enter bed time (t_{bed}), a go-to-sleep time (t_{GTS}), an initial sleep time (tₛₗₑₑₚ), a first micro-awakening MA₁ and a second micro-awakening MA₂, a wake-up time (t_{wake}), and a rising time (tᵣᵢₛₑ).

As used herein, a sleep session can be defined in multiple ways. For example, a sleep session can be defined by an initial start time and an end time. In some implementations, a sleep session is a duration where the user is asleep, that is, the sleep session has a start time and an end time, and during the sleep session, the user does not wake until the end time. That is, any period of the user being awake is not included in a sleep session. From this first definition of sleep session, if the user wakes ups and falls asleep multiple times in the same night, each of the sleep intervals separated by an awake interval is a sleep session.

Alternatively, in some implementations, a sleep session has a start time and an end time, and during the sleep session, the user can wake up, without the sleep session ending, so long as a continuous duration that the user is awake is below an awake duration threshold. The awake duration threshold can be defined as a percentage of a sleep session. The awake duration threshold can be, for example, about twenty percent of the sleep session, about fifteen percent of the sleep session duration, about ten percent of the sleep session duration, about five percent of the sleep session duration, about two percent of the sleep session duration, etc., or any other threshold percentage. In some implementations, the awake duration threshold is defined as a fixed amount of time, such as, for example, about one hour, about thirty minutes, about fifteen minutes, about ten minutes, about five minutes, about two minutes, etc., or any other amount of time.

In some implementations, a sleep session is defined as the entire time between the time in the evening at which the user first entered the bed, and the time the next morning when user last left the bed. Put another way, a sleep session can be defined as a period of time that begins on a first date (e.g., Monday, Jan. 6, 2020) at a first time (e.g., 10:00 PM), that can be referred to as the current evening, when the user first enters a bed with the intention of going to sleep (e.g., not if the user intends to first watch television or play with a smart phone before going to sleep, etc.), and ends on a second date (e.g., Tuesday, Jan. 7, 2020) at a second time (e.g., 7:00 AM), that can be referred to as the next morning, when the user first exits the bed with the intention of not going back to sleep that next morning.

In some implementations, the user can manually define the beginning of a sleep session and/or manually terminate a sleep session. For example, the user can select (e.g., by clicking or tapping) one or more user-selectable element that is displayed on the display device 172 of the user device 170 (FIG. 1) to manually initiate or terminate the sleep session.

Referring to FIG. 3, an exemplary timeline 300 for a sleep session is illustrated. The timeline 300 includes an enter bed time (t_{bed}), a go-to-sleep time (t_{GTS}), an initial sleep time (tₛₗₑₑₚ), a first micro-awakening MA₁, a second micro-awakening MA₂, an awakening A, a wake-up time (t_{wake}), and a rising time (tᵣᵢₛₑ).

The enter bed time t_{bed} is associated with the time that the user initially enters the bed (e.g., bed 230 in FIG. 2) prior to falling asleep (e.g., when the user lies down or sits in the bed). The enter bed time t_{bed} can be identified based on a bed threshold duration to distinguish between times when the user enters the bed for sleep and when the user enters the bed for other reasons (e.g., to watch TV). For example, the bed threshold duration can be at least about 10 minutes, at least about 20 minutes, at least about 30 minutes, at least about 45 minutes, at least about 1 hour, at least about 2 hours, etc. While the enter bed time t_{bed} is described herein in reference to a bed, more generally, the enter time t_{bed} can refer to the time the user initially enters any location for sleeping (e.g., a couch, a chair, a sleeping bag, etc.).

The go-to-sleep time (GTS) is associated with the time that the user initially attempts to fall asleep after entering the bed (t_{bed}). For example, after entering the bed, the user may engage in one or more activities to wind down prior to trying to sleep (e.g., reading, watching TV, listening to music, using the user device 170, etc.). The initial sleep time (tₛₗₑₑₚ) is the time that the user initially falls asleep. For example, the initial sleep time (tₛₗₑₑₚ) can be the time that the user initially enters the first non-REM sleep stage.

The wake-up time t_{wake} is the time associated with the time when the user wakes up without going back to sleep (e.g., as opposed to the user waking up in the middle of the night and going back to sleep). The user may experience one of more unconscious microawakenings (e.g., microawakenings MA₁ and MA₂) having a short duration (e.g., 5 seconds, 10 seconds, 30 seconds, 1 minute, etc.) after initially falling asleep. In contrast to the wake-up time t_{wake}, the user goes back to sleep after each of the microawakenings MA₁ and MA₂. Similarly, the user may have one or more conscious awakenings (e.g., awakening A) after initially falling asleep (e.g., getting up to go to the bathroom, attending to children or pets, sleep walking, etc.). However, the user goes back to sleep after the awakening A. Thus, the wake-up time t_{wake} can be defined, for example, based on a wake threshold duration (e.g., the user is awake for at least 15 minutes, at least 20 minutes, at least 30 minutes, at least 1 hour, etc.).

Similarly, the rising time tᵣᵢₛₑ is associated with the time when the user exits the bed and stays out of the bed with the intent to end the sleep session (e.g., as opposed to the user getting up during the night to go to the bathroom, to attend to children or pets, sleep walking, etc.). In other words, the rising time tᵣᵢₛₑ is the time when the user last leaves the bed without returning to the bed until a next sleep session (e.g., the following evening). Thus, the rising time tᵣᵢₛₑ can be defined, for example, based on a rise threshold duration (e.g., the user has left the bed for at least 15 minutes, at least 20 minutes, at least 30 minutes, at least 1 hour, etc.). The enter bed time t_{bed} time for a second, subsequent sleep session can also be defined based on a rise threshold duration (e.g., the user has left the bed for at least 4 hours, at least 6 hours, at least 8 hours, at least 12 hours, etc.).

As described above, the user may wake up and get out of bed one more times during the night between the initial t_{bed} and the final tᵣᵢₛₑ. In some implementations, the final wake-up time t_{wake} and/or the final rising time tᵣᵢₛₑ that are identified or determined based on a predetermined threshold duration of time subsequent to an event (e.g., falling asleep or leaving the bed). Such a threshold duration can be customized for the user. For a standard user which goes to bed in the evening, then wakes up and goes out of bed in the morning any period (between the user waking up (t_{wake}) or raising up (tᵣᵢₛₑ), and the user either going to bed (t_{bed}), going to sleep (t_{GTS}) or falling asleep (tₛₗₑₑₚ) of between about 12 and about 18 hours can be used. For users that spend longer periods of time in bed, shorter threshold periods may be used (e.g., between about 8 hours and about 14 hours). The threshold period may be initially selected and/or later adjusted based on the system monitoring the user's sleep behavior.

The total time in bed (TIB) is the duration of time between the time enter bed time t_{bed} and the rising time tᵣᵢₛₑ. The total sleep time (TST) is associated with the duration between the initial sleep time and the wake-up time, excluding any conscious or unconscious awakenings and/or micro-awakenings therebetween. Generally, the total sleep time (TST) will be shorter than the total time in bed (TIB) (e.g., one minute short, ten minutes shorter, one hour shorter, etc.). For example, referring to the timeline 300 of FIG. 3, the total sleep time (TST) spans between the initial sleep time tₛₗₑₑₚ and the wake-up time t_{wake}, but excludes the duration of the first micro-awakening MA₁, the second micro-awakening MA₂, and the awakening A. As shown, in this example, the total sleep time (TST) is shorter than the total time in bed (TIB).

In some implementations, the total sleep time (TST) can be defined as a persistent total sleep time (PTST). In such implementations, the persistent total sleep time excludes a predetermined initial portion or period of the first non-REM stage (e.g., light sleep stage). For example, the predetermined initial portion can be between about 30 seconds and about 20 minutes, between about 1 minute and about 10 minutes, between about 3 minutes and about 5 minutes, etc. The persistent total sleep time is a measure of sustained sleep, and smooths the sleep-wake hypnogram. For example, when the user is initially falling asleep, the user may be in the first non-REM stage for a very short time (e.g., about 30 seconds), then back into the wakefulness stage for a short period (e.g., one minute), and then goes back to the first non-REM stage. In this example, the persistent total sleep time excludes the first instance (e.g., about 30 seconds) of the first non-REM stage.

In some implementations, the sleep session is defined as starting at the enter bed time (t_{bed}) and ending at the rising time (tᵣᵢₛₑ), i.e., the sleep session is defined as the total time in bed (TIB). In some implementations, a sleep session is defined as starting at the initial sleep time (tₛₗₑₑₚ) and ending at the wake-up time (t_{wake}). In some implementations, the sleep session is defined as the total sleep time (TST). In some implementations, a sleep session is defined as starting at the go-to-sleep time (t_{GTS}) and ending at the wake-up time (t_{wake}). In some implementations, a sleep session is defined as starting at the go-to-sleep time (t_{GTS}) and ending at the rising time (tᵣᵢₛₑ). In some implementations, a sleep session is defined as starting at the enter bed time (t_{bed}) and ending at the wake-up time (t_{wake}). In some implementations, a sleep session is defined as starting at the initial sleep time (tₛₗₑₑₚ) and ending at the rising time (tᵣᵢₛₑ).

In some implementations, one or more of the sensors 130 can be used to determine or identify the enter bed time (t_{bed}), the go-to-sleep time (t_{GTS}), the initial sleep time (tₛₗₑₑₚ), one or more first micro-awakenings (e.g., MA₁ and MA₂), the wake-up time (t_{wake}), the rising time (tᵣᵢₛₑ), or any combination thereof, which in turn define the sleep session. For example, the enter bed time t_{bed} can be determined based on, for example, data generated by the motion sensor 138, the microphone 140, the camera 150, or any combination thereof. The go-to-sleep time can be determined based on, for example, data from the motion sensor 138 (e.g., data indicative of no movement by the user), data from the camera 150 (e.g., data indicative of no movement by the user and/or that the user has turned off the lights) data from the microphone 140 (e.g., data indicative of the using turning off a TV), data from the user device 170 (e.g., data indicative of the user no longer using the user device 170), data from the pressure sensor 132 and/or the flow rate sensor 134, or any combination thereof.

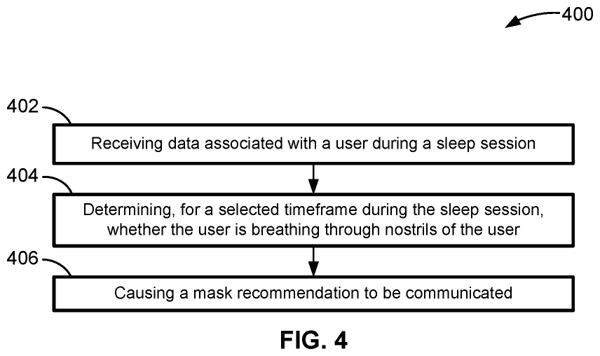

Referring to FIG. 4, a method 400 for determining a mask recommendation for a user is illustrated, according to some implementations of the present disclosure. One or more steps of the method 400 can be implemented using any element or aspect of the system 100 (FIGS. 1-2) described herein.

Step 402 of the method 400 includes receiving and/or generating data associated with a user during a sleep session. The data can include, for example, respiration data associated with the user, audio data associated with the user, or both the respiration data and the audio data. The respiration data is indicative of respiration of the user (e.g., a respiration rate, a respiration rate variability, a tidal volume, an inspiration amplitude, an expiration amplitude, and/or an inspiration-expiration ratio) during at least a portion of the sleep session (e.g., at least 10% of the sleep session, at least 50% of the sleep session, 75% of the sleep session, at least 90% of the sleep session, etc.). The audio data is reproducible as one or more sounds recorded during the sleep session (e.g., snoring, coughing, choking, breathing, a pause in breathing, labored breathing, etc.).

In some implementations, the respiration data is generated by a first one of the one or more sensors 130 and the audio data is generated by a second one of the one or more sensors 130. For example, the respiration data can be generated by the temperature sensor 136, the pressure sensor 130 and/or flow rate sensor 132 and the audio data can be generated by the microphone 140. In this example, the pressure sensor 130 and/or the flow rate sensor 132 can be coupled to or integrated in any component or aspect of the substrate 190. The microphone 140 can be coupled to or integrated in the user device 170 and/or the substrate 190. In other implementations, the respiration data and the audio data are generated by the same one(s) of the one or more sensors 130. In such implementations, the respiration data and the audio data can be generated by, for example, the acoustic sensor 141. The data can be received from the one or more sensors 130 by, for example, the electronic interface 119 and/or the user device 170 (FIG. 1) described herein.

The respiration data and the audio data can be timestamped such that a portion of the audio data can be associated with a corresponding portion of the respiration data that is associated with the same time interval. The moisture sensor 176 can be used to sense humidity in the air breathed by the user. The motion sensor 138 can be used to determine whether the user is awake and/or can be used to determine a sleeping position of the user.

Step 404 of the method 400 includes determining, for a selected timeframe during the sleep session, whether the user is breathing through the nostrils of the user based at least in part on the data received during step 402. Detecting whether the user is breathing through the nose or mouth for the selected timeframe can be determined based at least in part on the respiration data, the audio data, moisture data, etc. For example, the control system 110 can analyze the data (e.g., that is stored in the memory device 114) received during step 402 to determine the respiration signal associated with the user during the sleep session. Information associated with and/or describing the determined respiration signal can be stored in the memory device 114 (FIG. 1), for example.

The selected timeframe can be a percentage of the sleep session (e.g., 90% of the sleep session, 80% of the sleep session, 60% of the sleep session, etc.). The selected timeframe can be a total period of time during the sleep session that the user is asleep. That is, the selected timeframe can be the TST as defined in FIG. 3. In some implementations, the selected timeframe is adjusted to remove periods where the user experienced an apnea event. In some implementations, the user has a calculated AHI value, and the selected timeframe is adjusted based on the AHI value. Adjustment based on AHI value can include estimating an amount of time for an apnea event and multiplying this number by the AHI value to obtain an hourly correction to be made to the selected timeframe. The hourly correction can be prorated based on the duration of the selected timeframe. For example, if the selected timeframe is two hours, then the hourly correction is multiplied by two to obtain the prorated correction. If the selected timeframe is 6.5 hours, then the hourly correction is multiplied by 6.5 to obtain the prorated correction. If the selected timeframe is forty-five minutes (or 0.75 hours), then the hourly correction is multiplied by 0.75 to obtain the prorated correction.

In some implementations, the user does not breathe through her nose the entirety of the selected timeframe or the user alternates between breathing through her nose and breathing through her mouth. Or in some implementations, the user breaths through her mouth for the entirety of the selected timeframe. As such, a nose-breathing percent can be determined for the selected timeframe. The nose-breathing percent being a percentage of the selected timeframe that the user breathes through the nose. If the nose-breathing percent is greater than a threshold, then the user is determined to be a nose-breather. In some implementations, the threshold is 50%, 60%, 70%, 90%, etc. If the nose-breathing percent is less than or equal to the threshold, then the user is determined to be a mouth-breather. The nose-breather is an individual with a breathing habit of breathing through the nose, while a mouth-breather is an individual with a breathing habit of breathing through the mouth.

In some implementations, the threshold can be adjusted based on the duration of the selected timeframe. For example, if the selected timeframe is on the order of minutes, then the threshold can on the higher end (e.g., around 75%, 80%, or 90%). If the selected timeframe is on the order of hours, then the threshold can be on the lower end (e.g., around 50%, 60%, 70%). In some implementations, if the selected timeframe is on the order of hours, then the threshold is on the higher end. The threshold being on the higher end when the selected timeframe is on the order of hours indicates that a higher portion of the sleep session is being captured, hence is more representative of the user's sleeping habits when compared to a shorter timeframe on the order of minutes.

Determining the difference between nose-breathing and mouth-breathing can be based on location. For example, the substrate 190 can be positioned proximate to the nose to monitor airflow from the nose. If no airflow is observed from the nose within a period of time, then an assumption for mouth-breathing can be made. The substrate 190 can monitor moisture levels around the nostrils of the user. That way, exhalation can be detected since exhaled air can have a higher humidity than inhaled air in some implementations. In some implementations, the flow rate sensor 134 and/or the pressure sensor 132 is a flexible membrane that deviates when airflow is detected. When the membrane is positioned proximate to the nose, the deviation of the membrane can indicate nose-breathing.

In some implementations, the membrane is a piezoelectric layer that changes resistance based on deviation. As such, the deviation can be used to further distinguish whether the user is inhaling/exhaling through her nose or through both her nose and her mouth. For example, the membrane in a rest position indicates a first resistance level, and the membrane deviating reduces resistance of the membrane from the first resistance level. The reduction in resistance can indicate breathing through the nose. A second resistance level that is lower than the first resistance level can indicate that the user is breathing through only the nostrils. If the membrane changes resistance from the first resistance level to a resistance value between the first resistance level and the second resistance level, then the user is determined to be breathing through both her nose and mouth. If the membrane changes resistance from the first resistance level to a resistance value lower than or equal to the second resistance level, then the user is determined to be breathing through the nostrils only.

In some implementations, a breathing pattern of the user can be determined based on the data received at step 402. The breathing pattern of the user can include (a) inhaling through the nostrils, inhaling through the mouth, or both, and (b) exhaling through the nostrils, exhaling through the mouth, or both. In some implementations, the breathing pattern is determined based on a change in airflow direction through the nostrils. In some implementations, the breathing pattern is determined based on a change in humidity. For example, sensing humidity above a humidity threshold and then sensing humidity below the humidity threshold within a period of time can indicate inspiration followed by exhalation. In some implementations, sensing consecutive exhalations or expirations through the nostrils without inhalations through the nostrils can indicate mouth inhalations. Consecutive inhalations through the nostrils without exhalations through the nostrils can indicate mouth exhalations.

In some implementations, pressure levels can be used to determine breathing pattern. For example, the substrate 190 can determine an ambient pressure level during a gap in breathing. The ambient pressure level indicates a level where the user is neither exhaling or inhaling. During expiration, the substrate 190 can sense a first pressure level higher than the ambient pressure level and can determine that the user is exhaling. During inspiration, the substrate 190 can sense a second pressure level below the ambient pressure level and can determine that the user is inhaling. In some implementations, the substrate 190 can sense a third pressure level between the first pressure level and the ambient pressure level and determine that the user is partly exhaling through the nostrils. The substrate 190 can sense a fourth pressure level between the ambient pressure level and the second pressure level and determine that the user is partly inhaling through the nostrils. Partly exhaling and partly inhaling can be interpreted as the user exhaling using both the nose and mouth and the user inhaling using both the nose and mouth, respectively.

In some implementations, the motion sensor 138 and/or the force sensor 162 is used to determine one or more sleeping positions of the user. The sleeping positions include a supine sleeping position, a prone sleeping position, a left side sleeping position, a right side sleeping position, a sleeping position where a head of the user is elevated, or any combination thereof. The breathing habit of the user can be different for different sleeping positions, as such, time stamping can be used to associate breathing patterns with sleeping positions of the user during the selected timeframe. In some implementations, the user is a nose-breather in certain sleeping positions and a mouth-breather in other sleeping positions. In some implementations, the force sensor 162 is coupled to a gyroscope and/or an accelerometer on the substrate 190 such that determination of the position of the substrate 190 in space is used to approximate the sleeping positions of the user. Since the substrate 190 is attached to the nose of the user, the position of the substrate 190 in space translates to the position of the head of the user. Thus, the force sensor 162 and/or the motion sensor 138 can be used to not only detect sleeping positions but head and/or neck positions based on the position of the substrate 190.

In some implementations, a congestion level of the user can be determined using ultrasonic pulse data, laser light data, or both. The ultrasonic pulse data, laser light data, or both can be used to image the nasal cavity of the user to determine whether there is a blockage within the nasal cavity. If the congestion level is greater than a congestion threshold, then the received data for the sleep session is regarded as unreliable. The congestion threshold can be defined as a distance that ultrasonic pulse data can travel prior to reaching a boundary. In some implementations, the user is determined to have a respiratory illness (e.g., the user is determined to have a cold). The user may be informed to provide data from a future sleep session when the user is less congested.

In some implementations, a mask size recommendation can be determined using ultrasonic pulse data, laser light data, or both. Mask sizes are typically based on height and width of the nose of the user. The ultrasonic pulse data, laser light data, or both can be used to image the nasal cavity of the user. The control system 110 can use the image of the nasal cavity to reconstruct a potential image of the shape of the nose of the user in order to approximate the height and width of the nose of the user. Based on the approximated height and width of the nose of the user, a mask size recommendation can be determined using a look-up table or a mask sizing chart. In an example, nasal masks with a petite size are recommended for noses that are about 1.5 inches tall and 1.5 inches wide, nasal masks with a small size are recommended for noses that are about 1.75 inches tall and 1.5 inches wide, nasal masks with a medium-small size are recommended for noses that are about 2 inches tall and 1.5 inches wide, nasal masks with a large size are recommended for noses that are about 2.25 inches tall and 2 inches wide, etc.

In some implementations, the mask size recommendation can be determined using the user device 170. The camera 150 coupled to the user device 170 can be used to capture image data of the face of the user. In an example, the user device 170 is a smartphone of the user and the camera 150 includes at least two cameras such that a defined width between the two cameras indicates a unit length for determining sizes of objects in images captured by the camera 150. The control system 110 can use object detection algorithms to identify the nose and mouth of the user in the image data of the face of the user. Based on the height and width of the nose of the user, the mask size recommendation can be determined using a look-up table as previously described in connection with nasal masks. In some implementations, a full face mask can be recommended by determining dimensions of a height of the combined nose and mouth area of the user and a width of the mouth of the user. A full face mask with a small size can be recommended for a height of 3.25 inches and width of 2.75 inches. A full face mask with a medium size can be recommended for a height of 3.5 inches and a width of 3.25 inches. A full face mask with a large size can be recommended for a height of 4.25 inches and a width of 3.25 inches.

Step 406 of the method 400 includes causing a mask recommendation to be communicated. The mask recommendation can be communicated to a doctor of the user, a caretaker of the user, a partner of the user, or any combination thereof. The mask recommendation can be provided by the user device 170 based on data generated by the substrate 190. The mask recommendation can be indicated as a visual signal including a light provided on the substrate 190. For example, an LED can be provided on the substrate 190 such that the recommendation of a full facial mask is a red light, and the recommendation for a nasal mask is a green light. In some implementations, the LED lights up only for a nasal mask or only for a full facial mask. Nasal mask and full facial mask are used as examples, but a nasal pillow can be provided as well.

In some implementations, the mask recommendation can include an auditory signal, a tactile signal, etc. The auditory signal can be provided by the speaker 142. The speaker 142 can be provided on the user device 172. The user device 172 can be a laptop computer, a smart phone, a smart speaker, etc.

In some implementations, the mask recommendation includes the breathing pattern of the user. In some implementations, the mask recommendation includes the breathing habit of the user. In some implementations, the mask recommendation includes sleeping positions of the user. In some implementations, the mask recommendation includes recommended sleeping positions of the user. The recommended sleeping positions of the user can be positions where the breathing habit of the user is a nose-breathing habit. In some implementations, the mask recommendation includes a mask size recommendation for the user.

In some implementations, the substrate 190 includes uses the motion sensor 138 to determine that the user has coupled the substrate 190 to her nostrils. The substrate 190 can turn on based on increased activity of the motion sensor 138. In some implementations, the substrate 190 includes a membrane with resistivity that changes based on whether the membrane is exposed to a humid environment. Air from nostrils when the user exhales is more humid than typical sealed environment of an unused substrate 190. As such, the change in humidity can be used to determine that the substrate 190 is coupled to the nostrils of the user. In some implementations, the substrate 190 includes the temperature sensor 136 for determining that the substrate 190 is coupled to the nostrils of the user. Temperature of the packaging of the substrate can be lower than temperature of the human body.

Once determined that the substrate 190 is coupled to the nostrils of the user using the motion sensor 138, the moisture sensor 176, the temperature sensor 136, and/or any of the one or more sensors 130, electronics provided on the substrate 190 can be turned on to a monitoring state such that physiological data relating to the user's respiration can be collected. In some implementations, a tab is removed from the substrate 190, such that when the tab is removed, the substrate 190 is placed in the monitoring state. In some implementations, the substrate 190 includes an on/off button for placing the substrate 190 into the monitoring state.

In some implementations, a machine learning algorithm that is trained (e.g., using supervised or unsupervised learning) using received respiration data (step 402) to determine whether the user is breathing through the nostrils (step 404). The received respiration data can be calibrated using instructions provided to the user. For example, the substrate 190 can be calibrated prior to the user using the substrate 190 during the sleep session. The user device 170 can provide an instruction to the user to perform at least one in-and-out breath pair. An in-and-out breath pair includes the user breathing in through the nostrils and breathing out through the nostrils. The substrate 190 can generate data associated with each in-and-out breath pair to determine a nose-breathing baseline for the user. The nose-breathing baseline can include a baseline inspiration flow rate, a baseline expiration flow rate, a baseline moisture level, a baseline breathing sound volume, or any combination thereof. As such, during step 404, the nose-breathing baseline can be compared with respiration data of step 402 during the sleep session to determine whether the respiration data deviates from the nose-breathing baseline. For example, if the respiration data deviates from the nose-breathing data, then the user can be determined to be partially breathing from the nose or not breathing at all from the nose.

In some implementations, the substrate 190 can use red, amber, green visual alerts to indicate whether the user is a nose-breather. For example, a nose-breather can be indicated with an LED that lights up as green, indicating that the user should be recommended a nasal or pillow mask. Amber can indicate that the user sometimes is a nose-breather and sometimes is a mouth-breather. That is, during the selected time-frame, if a magnitude of the nose-breathing percent is comparable to a mouth-breathing percent, then an amber can indicate that the user can learn to use a nasal or pillow mask but should be monitored closely. Red can indicate that the user should start with a full face mask since the user is not a nose-breather. These colors are used merely as examples. In some implementations, more than three colors can be used or a different color scheme can be used. For example, a color scheme or theme for those suffering from color blindness can be provided.

In some implementations, the substrate 190 can be used alongside the user interface 124 during therapy. The substrate 190 can monitor during therapy whether the user has switched from a mouth-breather to a nose-breather. As such, if the user is compliant with a full face mask for weeks and is trending towards being a nose-breather, then a recommendation for a nasal or pillow mask can be recommended. The benefit of recommending a smaller mask can be useful for users who may feel claustrophobic when using a full face mask. This can improve adherence to therapy and improve compliance.

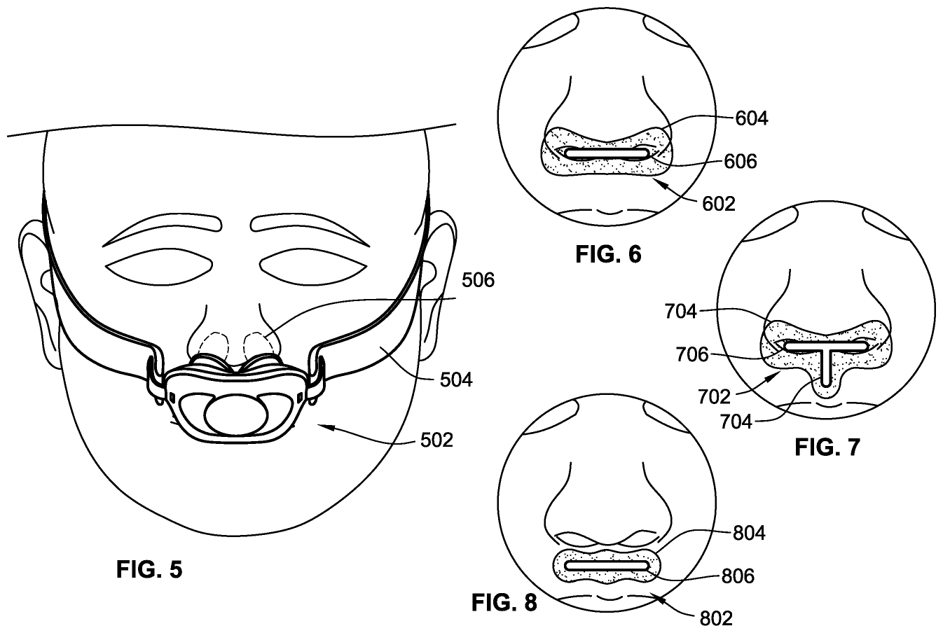

FIG. 5 illustrates an example of a substrate 502 according to some implementations of the present disclosure. The substrate 502 is similar to or the same as the substrate 190 of FIG. 1. The substrate 502 includes conical portions 506 that are inserted into the nostrils as shown in FIG. 5. The substrate 502 is held in place by straps 504 so that the substrate 502 remains affixed to the nose while the user is asleep. The straps 504 are depicted in FIG. 5 as looping around the head of the user. In some implementations, the straps 504 can include chin straps where pieces of the straps 504 also loop around the chin of the user. The one or more sensors 130 can be provided on the substrate 502. For example, a membrane can be provided for pressure measurements, flow rate measurements, etc. A battery (e.g., a button cell) can be used to power electronics included in the substrate 502.

FIG. 6 illustrates an example of a substrate 602, according to some implementations of the present disclosure. The substrate 602 is similar to or the same as the substrate 190 of FIG. 1. The substrate 602 is affixed to the nose using adhesive. A portion 604 of the substrate 602 can include the adhesive for keeping a sensing portion 606 of the substrate 602 across both nostrils of the user.

FIG. 7 illustrates an example of a substrate 702, according to some implementations of the present disclosure. The substrate 702 is similar to or the same as the substrate 190 of FIG. 1. Similar to the substrate 602, the substrate 702 is affixed to the user using adhesive on a portion 704. The substrate 702 includes two sensing portions 706 and 708. The sensing portion 706 can be used to determine nose-breathing, and the sensing portion 708 can be used to monitor movement of the lips to increase an accuracy for determining whether the user is mouth-breathing. In some implementations, the sensing portions 706 and 708 include microphones such that breathing sounds from the nose can be distinguished from breathing sounds from the mouth. For example, one microphone can be located closer to the nose on the sensing portion 706 and a second microphone can be located closer to the mouth on the sensing portion 708. Differential loudness from both microphones can be used to determine whether the user is nose-breathing or mouth-breathing.

FIG. 8 illustrates an example of a substrate 802, according to some implementations of the present disclosure. The substrate 802 is similar to or the same as the substrate 190 of FIG. 1 and the substrate 602 of FIG. 6. In FIG. 8, the substrate 802 is designed to be affixed below (i.e., adjacent to the nose). The substrate 802 includes a portion 804 with adhesive to affix the substrate 802 to the user. A sensing portion 806 is provided on the substrate 802 to contain electronics and/or one or more of the sensors 130 for capturing respiration data.

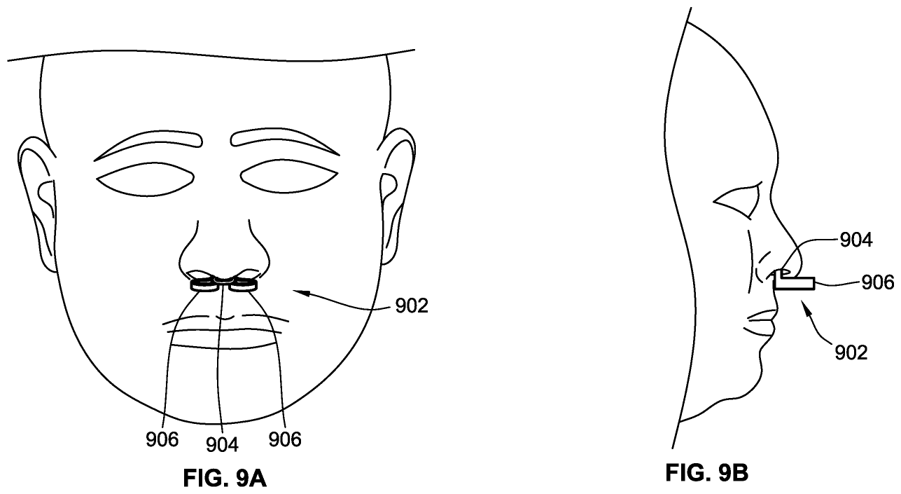

FIG. 9A shows a front view of an example substrate 902, according to some implementations of the present disclosure. FIG. 9B shows a side view of the substrate 902. The substrate 902 is similar to or the same as the substrate 190 of FIG. 1. The substrate 902 includes a clip 904 at attaches to the septum. The clip 904 holds the substrate 902 in place while the user is asleep. The substrate 902 includes sensing portions 906 which include electronics and/or one or more of the sensors 130 for capturing respiration data. In some implementations, the clip 904 includes a wire that connects both sensing portions 906 such that power from a battery can be shared between electronics on the sensing portions 906.

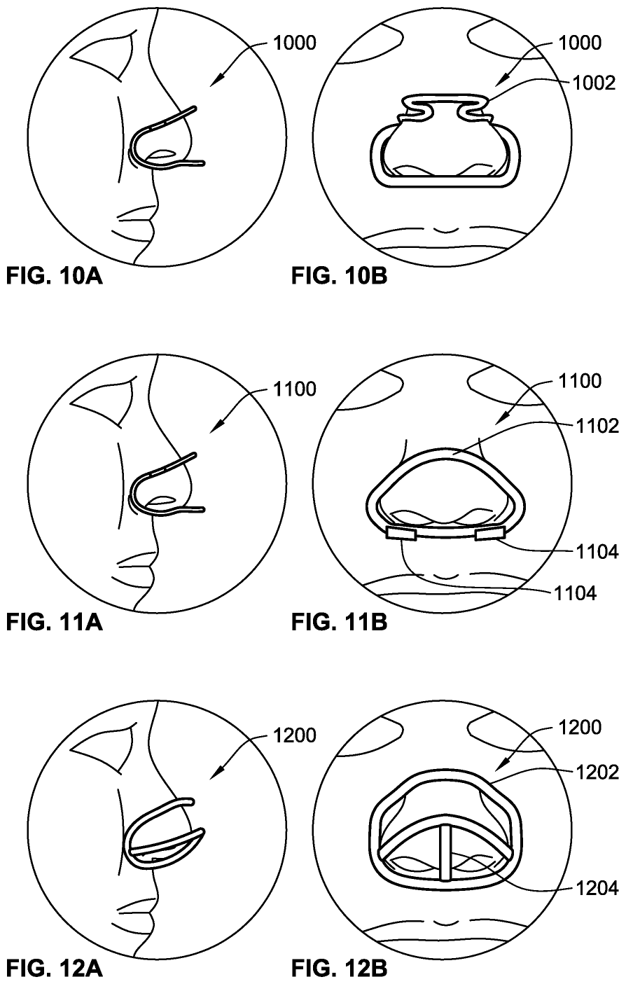

FIG. 10A shows a side view of a substrate 1000 which is similar to or the same as the substrate 190, according to some implementations of the present disclosure. FIG. 10B shows a front view of the substrate 1000. The substrate 1000 includes a frame 1002 that pinches around the nasal bridge of the user to keep the substrate 1000 in place.

FIG. 11A shows a side view of a substrate 1100 which is similar to or the same as the substrate 190, according to some implementations of the present disclosure. FIG. 11B shows a front view of the substrate 1100. The substrate 1100 includes a frame 1102 that hugs the nose of the user to keep sensing portions 1104 proximate to the nostrils of the user.

FIG. 12A shows a side view of a substrate 1200 which is similar to or the same as the substrate 190, according to some implementations of the present disclosure. FIG. 12B shows a front view of the substrate 1200. The substrate 1200 includes a cage 1202 that hugs the nose of the user. The cage 1202 can have a portion 1204 that separates both nostrils. The cage 1202 can hold a membrane and/or other electronics for capturing respiration data as discussed above.

FIGS. 5-12B are provided as examples of how to couple the substrate 190 (FIG. 1) to the user 210 (FIG. 2) in order to gather data. In some implementations, the substrate 190 can be incorporated into different items worn away from the nose of the user. For example, the substrate 190 can be incorporated in a head-mounted display (HMD). In another example, the substrate 190 can be incorporated into a sleep mask. A sleep mask is used to block out light from the environment while a user of the sleep mask is trying to sleep or while the user is asleep. The sleep mask covers the eyes of the user and can have a strap that goes around the user's head so that the sleep mask is held in place. The sleep mask can incorporate the substrate 190 and the one or more sensors 130 to obtain data while the user is asleep. In some implementations, the sleep mask includes lights and speakers that helps the user sleep.

In another example, the substrate 190 can be incorporated in earbuds and/or earplugs. The user can sleep with the earbuds, and sensors on the substrate 190 can collect sleep data, according to some implementations of the present disclosure. For example, the substrate 190 can use the microphone 140 (FIG. 1) to listen for sounds to detect congestion, breathing sounds, breathing rate, apneas, snoring, mouth breathing, nasal breathing, head position, etc. In some implementations, head position is easier to detect because when two earbuds are used, microphones on one earbud being muffled indicates that the user's head is resting sideways. When none of the earbuds is muffled, then the back of the user's head is resting against the bed.

One or more elements or aspects or steps, or any portion(s) thereof, from one or more of any of claims 1-44 below can be combined with one or more elements or aspects or steps, or any portion(s) thereof, from one or more of any of the other claims 1-44 or combinations thereof, to form one or more additional implementations and/or claims of the present disclosure.

While the present disclosure has been described with reference to one or more particular embodiments or implementations, those skilled in the art will recognize that many changes may be made thereto without departing from the spirit and scope of the present disclosure. Each of these implementations and obvious variations thereof is contemplated as falling within the spirit and scope of the present disclosure. It is also contemplated that additional implementations according to aspects of the present disclosure may combine any number of features from any of the implementations described herein.

1. A method comprising:
receiving data associated with a user during a sleep session;
analyzing the received data to determine, for a selected timeframe during the sleep session, whether the user is breathing through nostrils of the user; and
based at least in part on a result of the analysis, causing a mask recommendation to be communicated.

2. The method of claim 1, wherein the selected timeframe is at least about 90 percent of the sleep session.

3. The method of claim 1, wherein the selected timeframe is a total period of time during the sleep session that the user is asleep.

4. The method of claim 1, wherein a duration of the selected timeframe is adjusted based at least in part on an apnea hypopnea index (AHI) value for the user.

5. The method of claim 1, wherein the mask recommendation indicates a breathing habit of the user, the breathing habit of the user being one of a mouth-breathing habit or a nose-breathing habit.

6-8. (canceled)

9. The method of claim 1, further comprising:
determining a nose-breathing percent for the selected timeframe indicating a percentage of the selected timeframe that the user breathed through the nostrils; and
determining that the user is a nose-breather based at least in part on the nose-breathing percent for the selected timeframe exceeding a threshold; and
determining that the user is a mouth-breather based at least in part on the nose-breathing percent for the percent of time being below the threshold.

10. (canceled)

11. The method of claim 1, further comprising:
determining, from the received data, a breathing pattern of the user, wherein the breathing pattern includes (a) inhaling through the nostrils, inhaling through a mouth of the user, or both, and (b) exhaling through the nostrils, exhaling through the mouth, or both;
wherein the mask recommendation indicates the breathing pattern of the user.

12. The method of claim 11, wherein the breathing pattern is determined to include inhaling through the nostrils followed by exhaling through the nostrils based at least in part on the received data indicating a change in airflow direction through the nostrils.

13. The method of claim 11, wherein the breathing pattern is determined to include (i) inhaling through the nostrils followed by exhaling through the mouth, (ii) exhaling through the nostrils followed by inhaling through the mouth, (iii) inhaling through the mouth followed by exhaling through the nostrils, or (iv) exhaling through the mouth followed by inhaling through the nostrils, based at least in part on determining consecutive inhalations without expirations or consecutive expirations without inhalations through the nostrils.

14. The method of claim 11, wherein the breathing pattern is determined to include inhaling through both the nostrils and the mouth or exhaling through both the nostrils and the mouth based at least in part on a pressure level around the nostrils being below a threshold.

15. (canceled)

16. The method of claim 1, further comprising:
determining, from the received data, one or more sleeping positions of the user during the sleep session, the sleeping position including a supine sleep position, prone sleep position, left side sleep position, right side sleep position, a sleep position where a head of the user is elevated, or any combination thereof; and
determining, for each sleeping position, a corresponding breathing habit of the user.

17. The method of claim 16, wherein the mask recommendation further indicates a recommended sleeping position of the user as any of the sleeping positions where the corresponding breathing habit of the user is a nose-breathing habit.

18. The method of claim 1, further comprising:
causing an instruction to perform at least one in-and-out breath pair to be communicated to the user, wherein the in-and-out breath pair includes the user breathing in through the nostrils and breathing out through the nostrils;
receiving data associated with the at least one in-and-out breath pair; and
determining a nose-breathing baseline for the user based at least in part on the data associated with the at least one in-and-out breath pair;
wherein the mask recommendation is further based at least in part on the nose-breathing baseline for the user.

19-20. (canceled)

21. The method of claim 1, further comprising:
determining, from the received data, a congestion level of the user, wherein the mask recommendation further indicates that data from a subsequent sleep session is required based at least in part on the congestion level being above a congestion threshold.

22-27. (canceled)

28. The method of claim 1, wherein the data is received from a substrate coupled to the nostrils of the user, and the mask recommendation is a visual signal including a light that is provided on the substrate.

29-34. (canceled)

35. A system for determining a breathing habit of a user, comprising:
a substrate coupled to nostrils of the user;
a memory storing machine-readable instructions; and
a control system including one or more processors configured to execute the machine-readable instructions to:
receive, from the substrate, data associated with the user during a sleep session;
analyze the received data to determine, for a selected timeframe, whether the user is breathing through the nostrils of the user; and
based at least in part on a result of the analysis, causing a mask recommendation to be communicated to the user.

36. The system of claim 35, wherein the substrate includes a membrane that deflects based on the user breathing.

37. The system of claim 36, wherein the membrane is configured to measure a pressure associated with the user breathing.

38. The system of claim 36, wherein the membrane covers at least one of the nostrils of the user.

39. The system of claim 36, wherein the membrane is configured to determine whether the user is breathing through both nostrils or through one.

40. The system of claim 36, wherein the membrane is configured to measure a flow rate of air through the nostrils of the user.

41. The system of claim 35, wherein the mask recommendation includes a visual signal, an auditory signal, a tactile signal, or a combination thereof.

42. The system of claim 41, wherein the substrate includes a light for providing the visual signal.

43. The system of claim 35, wherein the substrate includes one or more sensors including a pressure sensor, a flow rate sensor, a temperature sensor, an acoustic sensor, an infrared sensor, a camera, a force sensor, a capacitive sensor, a piezoresistive sensor, a moisture sensor, an oxygen sensor, or any combination thereof.

44. The system of claim 35, wherein the substrate is coupled to an external device including a mobile phone associated with the user, a smart speaker associated with the user, a desktop computer associated with the user, a laptop computer associated with the user, or any combination thereof.
